(19) United States
(12) Patent Application Publication
(10) Pub. No.: US 2026/0216269 A1
(43) Pub. Date: Jul. 30, 2026

(54) weight loss formulation with enzyme inhibitor

(71) Applicant: Kedar Prasad, San Rafael, CA (US)

(72) Inventor: Kedar Prasad, San Rafael, CA (US)

(21) Appl. No.: 18/831,923

(22) Filed: Nov. 17, 2025

Related U.S. Application Data
(60) Provisional application No. 63/833,865, filed on Jan. 27, 2025.

(51) Int. Cl. A61K 36/258; A61K 31/121; A61K 31/198; A61K 31/58; A61K 31/704; A61K 36/28; A61K 36/29; A61K 36/48; A61K 36/515; A61K 36/67; A61K 36/71; A61K 36/744; A61K 38/39

(52) U.S. Cl. CPC A61K 36/258; A61K 31/121; A61K 31/198; A61K 31/58; A61K 31/704; A61K 36/28; A61K 36/29; A61K 36/48; A61K 36/515; A61K 36/67; A61K 36/71; A61K 36/744; A61K 38/39

(57) ABSTRACT

A weight loss formulation comprises: pomegranate extract, glutamine, ginsenosides from ginseng extract, curcumin, fruit extract of Gardenia jasminoides, Gentiana scabra root extract, berberine from Berberis asiatica root, fenugreek seed extract, kalonji seed extract, black pepper extract, and an enzyme inhibitor such as methanol extract from Coreopsis lanceolata.

### RELATED APPLICATIONS

This application claims priority to the Provisional Application No. 63/833,865 filed on Jan. 27, 2024 entitled “Weight Loss Formulation and Related System’.

### BACKGROUND OF THE INVENTION

Growing obesity is a major health problem in the USA and world-wide. Excess consumption of high caloric fatty foods, poor social interaction, lack of physical activity, psychological factors contribute to obesity. Obesity causes increased oxidative stress and chronic inflammation which reduces the flow of insulin from the pancreas to the blood by damaging insulin receptors that leads to diabetes type II and diabetic related complications such as heart disease, kidney disease, eye diseases and peripheral neuropathy. Therefore, reducing body weight gradually and safely is urgently needed. Several commercial products for reducing body weight are available.

FDA approved drug Ozempic, which increases the secretion of only glucagon-like peptide-1 (GLP-1) for the treatment of diabetes, is available. Now, it is being widely used for weight loss. It has side effects including nausea and vomiting.

In fact, most weight loss products in the marketplace utilize the weight loss properties of GLP-1. However, GLP-1 has two major issues. First, it produces acute toxicity with short term risks such as nausea, vomiting, upset stomach, and diarrhea, and long term effects that include pancreatitis and thyroid cancer. Secondly, GLP-1 degrades very rapidly, thus requiring daily dosing, including daily injections like Ozempic. Furthermore, all weight loss products in the marketplace are synthetic causing toxicity.

The present invention provides a formulation and system formulation that addresses the issues relating to GLP-1.

### SUMMARY OF THE INVENTION

In one embodiment, the present invention relates to a formulation comprising pomegranate extract, glutamine, ginsenosides from ginseng extract, curcumin, fruit extract of Gardenia jasminoides, Gentiana scabra root extract, berberine from Berberis asiatica root, fenugreek seed extract, and kalonji seed extract. In another embodiment, the formulation functions as a weight loss formulation. In yet another further embodiment, the amount of pomegranate in the formulation is from about 50 mg to about 300 mg. In still another embodiment, the amount of the glutamine in the formulation is from about 100 mg to about 500 mg. In a still yet another embodiment, the amount of the ginsenosides in the formulation is from about 50 mg to about 200 mg. In a further embodiment, the amount of said curcumin in the formulation is from about 50 mg to about 300 mg. In still a further embodiment, the amount of the fruit extract of Gardenia jasminoides in the formulation is from about 50 mg to about 300 mg. In yet a further embodiment, the amount of the Gentiana scabra root extract in the formulation is from about 25 mg to about 200 mg. In another further embodiment, the amount of the berberine from Berberis asiatica root in the formulation is from about 50 mg to about 300 mg. In another embodiment, the amount of the Fenugreek seed extract in the formulation is from about 100 mg to about 400 mg. In a further embodiment, the amount of Kalonji seed extract in the formulation is from about 100 mg to about 300 mg. The formulation of the present invention generates both glucagon-like peptide-1 (GLP-1) and glucose-dependent insulinotropic polypeptide (GIP) and its mechanism allows GIP to protect against toxicity of GLP-1.

In another embodiment, the present invention provides for a formulation comprising of pomegranate extract, glutamine, ginsenosides from ginseng extract, curcumin, fruit extract of Gardenia jasminoides, Gentiana scabra root extract, berberine from Berberis asiatica root, fenugreek seed extract, kalonji seed extract and collagen peptide.

In a further embodiment, the formulation functions as a weight loss product.

In another further embodiment, said pomegranate extract is from about 50 mg to about 300 mg, said glutamine is from about 100 mg to about 500 mg, said ginsenosides is from about 50 mg to about 200 mg, said curcumin is from about 50 mg to about 300 mg, said fruit extract of Gardenia jasminoides is from about 50 mg to about 300 mg, said Gentiana scabra root extract is from about 25 mg to about 200 mg, said berberineis from about 50 mg to about 300 mg, said fenugreek seed extract is from about 100 mg to about 400 mg, said kalonji seed extract is from about 100 mg to about 400 mg and said collagen peptides are from about 5 grams to about 10 grams.

In yet another embodiment, the formulation further comprises at least one carrier and the carrier is selected from the group consisting of: powder, liquid, pill, hard shell capsule, soft shell capsule, gel, tablet, ointment, solution and combinations thereof.

In still another embodiment, the present invention relates to a formulation comprising of pomegranate extract, glutamine, ginsenosides from ginseng extract, curcumin, fruit extract of Gardenia jasminoides, Gentiana scabra root extract, berberine from Berberis asiatica root, fenugreek seed extract, kalonji seed extract, black pepper extract and methanol extract of Coreopsis lanceolata.

In still yet another embodiment, said pomegranate extract is from about 50 mg to about 300 mg, said glutamine is from about 100 mg to about 500 mg, said ginsenosides is from about 50 mg to about 200 mg, said curcumin is from about 50 mg to about 300 mg, said fruit extract of Gardenia jasminoides is from about 50 mg to about 300 mg, said Gentiana scabra root extract is from about 25 mg to about 200 mg, said berberineis from about 50 mg to about 300 mg, said fenugreek seed extract is from about 100 mg to about 400 mg, said kalonji seed extract is from about 100 mg to about 400 mg, black pepper extract is from about 5 mg to about 10 mg, and said methanol extract of Coreopsis lanceolata is from about 50 micrograms to about 200 micrograms.

### DETAILED DESCRIPTION OF THE INVENTION

As required, detailed embodiments of the present invention are disclosed herein; however, it is to be understood that the disclosed embodiments are merely exemplary of the invention that may be embodied in various forms. The figures are not necessarily to scale, some features may be exaggerated to show details of particular components. Therefore, specific structural and functional details disclosed herein are not to be interpreted as limiting, but merely as a basis for the claims and as a representative basis for teaching one skilled in the art to variously employ the present invention.

Hormones that can Reduce Body Weight:

Glucagon-like peptide-1 (GLP-1) and glucose-dependent insulinotropic polypeptide (GIP) are two hormones secreted by the intestine. They are rapidly degraded by the enzyme DPP-4 (dipeptidyl peptidase-4). The half-life of GLP-1 is less than 2 minutes.

Enhancing the production of these hormones increases weight loss.
1. Both GLP-1 and GIP stimulate beta-cells of the pancreas to increase the production and release of insulin to the blood which lowers glucose; however, GLP-1 is more effective than GIP.
2. Increased secretion of GLP-1 suppresses appetite and causes weight loss
3. Increased levels of GLP-1 can cause nausea and vomiting, but GIP prevents them. Therefore, any agent or combination of agents that increases the production of these two hormones would be more effective than the agent which can enhance production of only one hormone and thereby, would be more effective in reducing blood glucose levels and weight loss.

Proposed weight loss formulation would stimulate secretion of both glucagon-like peptide-1 (GLP-1) and glucose-dependent insulinotropic polypeptide (GIP). Therefore, this formulation would be more effective in reducing glucose levels in the blood and weight loss without any toxicity than those agents which would enhance production of only GLP-1. In addition, two Aurvedic herbs which would cause weight loss by mechanisms other than GLP-1 or GIP are added. Therefore, the proposed formulation would gradually decrease body weight without any toxicity.

### Formula for Weight Loss:

1. Pomegranate: The extract of seeds pomegranate is very rich in polyphenols which stimulate the secretion of GLP-1 that increases the secretion of insulin from the pancreas to the blood. It also acts as a strong antioxidant and reduces inflammation. Pomegranate seed extract also improves heart health. Pomegranate juice reduces hunger, and therefore, would be useful in weight loss.
2. Glutamine: Glutamine is the most abundant amino acid which is made in the body as well as consumed from the diet. It increases the secretion of both GLP-1 and GIP and stimulates the secretion of insulin from the pancreas to the blood to regulate glucose level. Glutamine is also useful in weight loss. GIP would reduce any potential side effects of GLP-1.
3. Ginsenosides: Ginsenosides, the main active component of Panax ginseng, can stimulate the secretion of glucagon-like peptide-1 (GLP-1) and may also affect the secretion of glucose-dependent insulinotropic polypeptide (GIP). This compound reduces insulin resistance and hyperglycemia and improves cognitive function in mice by increasing the level of acetylcholine by inhibiting acetylcholinesterase activity in cholinergic neurons. Acetylcholine is responsible for storing memory. In addition, Ginsenosides protect cholinergic neurons from oxidative and inflammatory damage. It also improves insulin sensitivity and decreases glucose formation in the liver, and reduces obesity.
4. Curcumin: Curcumin treatment improves glucose control by increasing the secretion of GLP-1 and GIP. Curcumin also reduces oxidative stress, inflammation, and adipogenesis, enhancing energy that may in combination with other ingredients may contribute to weight loss.
5. Fruit extract of Gardenia jasminoides: Fruits of Gardenia have been used for thousands of years in China to treat diabetes. The fruit extract ingredients stimulate GLP-1 pathway, possess potent antioxidant and anti-inflammatory properties, promote insulin secretion from the pancreas to the blood, and improve insulin sensitivity, protect islet B cells from oxidative and inflammatory damage, and improve the function macro- and microvascular systems. It also causes weight loss.
6. Gentiana scabra root extract: The extract of this bitter tasting herb improves GLP-1 secretion causing increased release of insulin from the pancreas to the blood which decreases hyperglycemia in a type 2 diabetic mouse model Gentiopicroside, a major active component of the Gentiana scabra root extract, reduced body weight and visceral fat mass in humans by decreasing the expression of adipogenesis/lipogenesis-related genes.
7. Berberine from Berberis asiatica root: Berberine is an alkaloid extracted from Rhizoma coptidis which has been traditionally used in Chinese medicine to treat gastrointestinal infections. It stimulates secretion of GLP-1. Berberine also suppresses hunger and thereby contributes to weight loss. It improves beta cell function and thereby improves production and flow of insulin from the pancreas to the blood causing decreased levels of glucose in the blood.
8. Fenugreek seed extract: Fenugreek seeds, also known as Methi, are commonly used in Ayurvedic medicine to aid digestion, regulate blood sugar levels, and promote weight loss. They are rich in soluble fiber, which helps increase satiety, reduce appetite, and promote the excretion of fat, inhibit fat accumulation, and lower bad cholesterol (LDL).
9. Kalonji (Nigella sativa) seed extract. In summary, our formula would be much better than all 4 products listed for reducing weight and blood glucose levels gradually without toxicity. In obese patients with diabetes, our formula may cause hypoglycemia. Therefore, it is essential to monitor blood glucose levels frequently. If hypoglycemia occurs, consult your doctor immediately. These products should be taken under the supervision of your doctor. Kalonji, also called black seed, black cumin, causes weight loss by reducing appetite. It also inhibits glucose absorption from the gut.
10. Methanol extract of Coreopsis lanceolata: A concentration of 100 g/ml inhibits DPP-4 (dipeptidyl peptidase-4) enzyme activity by 87.2%. Since DPP-4 enzyme quickly destroys GLP-1, a concentration of 150 g/ml was added to GLP-1 Boost to prevent its degradation so that effectiveness of GLP-1 lasts for a long time. This herb is non-toxic in humans.
11. Black pepper extract (pipeline) is added to enhance absorption of Curcumin.

The present invention would reduce weight and blood glucose levels gradually without toxicity. In obese patients with diabetes, our formula may cause hypoglycemia. Therefore, it is essential to monitor blood glucose levels frequently. If hypoglycemia occurs, consult your doctor immediately. This formula should be taken under the supervision of your doctor.

In one of the embodiments of the present invention, Table 1, set forth below, represents the ingredients and amount for the weight loss formulation.

TABLE 1
Weight loss formula (daily dose)

| Ingredient: | Amount: | |
|---|---|---|
| Pomegranate Extract | 100 | mg |
| Glutamine | 300 | mg |
| Ginsenosides from Ginseng extract | 100 | mg |
| Curcumin | 100 | mg |
| Fruits extract of Gardenia jasminoides | 100 | mg |
| Gentiana scabra root extract | 50 | mg |
| Berberine from Berberis Asiatica root | 100 | mg |
| Fenugreek seed extract | 200 | mg |
| Kalonji seed extract | 200 | mg |

In another embodiment, Table 2 represents the ingredients and range amounts for the weight loss formulation.

TABLE 2
Weight loss Formulation

| Ingredient: | Range Amount: |
|---|---|
| Pomegranate Extract | from about 50 mg to about 300 mg |
| Glutamine | from about 100 mg to about 500 mg |
| Ginsenosides from Ginseng extract | from about 50 mg to about 200 mg |
| Curcumin | from about 50 mg to about 300 mg |
| Fruits extract of Gardenia jasminoides | from about 50 mg to about 300 mg |
| Gentiana scabra root extract | from about 25 mg to about 200 mg |
| Berberine from Berberis Asiatica root | from about 50 mg to about 300 mg |
| Fenugreek seed extract | from about 100 mg to about 400 mg |
| Kalonji seed extract | from about 100 mg to about 300 mg |

Herbs producing only GLP-1:
1. Pomegranate extract
2. Extract of Gardenia jasminoides
3. Gentiana scabra root extract
4. Berberine from Berberis Asiatica root
Herbs Producing both GLPI-1 and GIP:
1. Glutamine
2. Ginsenosides
3. Curcumin
Herbs reducing appetite and inhibiting glucose absorption from the gut without GLP-1 or GIP:
1. Fenugreek seeds
2 . . . Kalonji (Nigella sativa) seed extract
Additional benefits for these herbs:
Exhibits antioxidant and anti-inflammation activities;
Lowers bad cholesterol; and
Improves general health.

In a further embodiment, Table 3 represents the ingredients and range amounts for the weight loss formulation.

TABLE 3
Weight loss formula (daily dose)

| Ingredient: | Amount: | |
|---|---|---|
| Pomegranate Extract | 100 | mg |
| Glutamine | 300 | mg |
| Ginsenosides from Ginseng extract | 100 | mg |
| Curcumin | 100 | mg |
| Fruits extract of Gardenia jasminoides | 100 | mg |
| Gentiana scabra root extract | 50 | mg |
| Berberine from Berberis Asiatica root | 100 | mg |
| Fenugreek seed extract | 200 | mg |
| Kalonji seed extract | 200 | mg |
| Black pepper extract | 6 | mg |
| Methanol extract of Coreopsis lanceolata | 150 | g/ml |

In another embodiment, Table 4 represents the ingredients and range amounts for the weight loss formulation.

TABLE 4
Weight loss Formulation

| Ingredient: | Range Amount: |
|---|---|
| Pomegranate Extract | from about 50 mg to about 300 mg |
| Glutamine | from about 100 mg to about 500 mg |
| Ginsenosides from Ginseng extract | from about 50 mg to about 200 mg |
| Curcumin | from about 50 mg to about 300 mg |
| Fruits extract of Gardenia jasminoides | from about 50 mg to about 300 mg |
| Gentiana scabra root extract | from about 25 mg to about 200 mg |
| Berberine from Berberis Asiatica root | from about 50 mg to about 300 mg |
| Fenugreek seed extract | from about 100 mg to about 400 mg |
| Kalonji seed extract | from about 100 mg to about 300 mg |
| Black pepper extract | from about 5 mg to about 10 mg |
| Methanol extract of Coreopsis lanceolata | from about 100 g/ml to about 200 g/ml |

Among those benefits and improvements that have been disclosed, other objects and advantages of this invention will become apparent from the following description taken in conjunction with the accompanying drawings. The drawings constitute a part of this specification and include exemplary embodiments of the present invention and illustrate various objects and features thereof.

Numerous modifications and variations of the present invention are possible in light of the above teachings. It is therefore to be understood that within the scope of the attendant claims attached hereto, this invention may be practiced otherwise than as specifically disclosed herein.

What is claimed is:

1. A formulation comprising of pomegranate extract, glutamine, ginsenosides from ginseng extract, curcumin, fruit extract of Gardenia jasminoides, Gentiana scabra root extract, berberine from Berberis asiatica root, fenugreek seed extract, and kalonji seed extract.

2. The formulation of claim 1 wherein said formulation functions as a weight loss product.

3. The formulation of claim 1 further comprises collagen peptides.

4. The formulation of claim 1 wherein said pomegranate extract is from about 50 mg to about 300 mg.

5. The formulation of claim 1 wherein said glutamine is from about 100 mg to about 500 mg.

6. The formulation of claim 1 wherein said ginsenosides is from about 50 mg to about 200 mg.

7. The formulation of claim 1 wherein said curcumin is from about 50 mg to about 300 mg.

8. The formulation of claim 1 wherein said fruit extract of Gardenia jasminoides is from about 50 mg to about 300 mg.

9. The formulation of claim 1 wherein said Gentiana scabra root extract is from about 25 mg to about 200 mg.

10. The formulation of claim 1 wherein said berberineis from about 50 mg to about 300 mg.

11. The formulation of claim 1 wherein said fenugreek seed extract is from about 100 mg to about 400 mg.

12. The formulation of claim 1 wherein said kalonji seed extract is from about 100 mg to about 400 mg.

13. A formulation comprising of pomegranate extract, glutamine, ginsenosides from ginseng extract, curcumin, fruit extract of Gardenia jasminoides, Gentiana scabra root extract, berberine from Berberis asiatica root, fenugreek seed extract, kalonji seed extract and collagen peptide.

14. The formulation of claim 13 wherein said formulation functions as a weight loss product.

15. The formulation of claim 13 wherein said pomegranate extract is from about 50 mg to about 300 mg, said glutamine is from about 100 mg to about 500 mg, said ginsenosides is from about 50 mg to about 200 mg, said curcumin is from about 50 mg to about 300 mg, said fruit extract of Gardenia jasminoides is from about 50 mg to about 300 mg, said Gentiana scabra root extract is from about 25 mg to about 200 mg, said berberineis from about 50 mg to about 300 mg, said fenugreek seed extract is from about 100 mg to about 400 mg, said kalonji seed extract is from about 100 mg to about 400 mg and said collagen peptides are from about 5 g to about 10 g.

16. The formulation of claim 15 further comprising at least one carrier.

17. The formulation of claim 16 wherein said carrier is selected from the group consisting of: powder, liquid, pill, hard shell capsule, soft shell capsule, gel, tablet, ointment, solution and combinations thereof.

18. A formulation comprising of pomegranate extract, glutamine, ginsenosides from ginseng extract, curcumin, fruit extract of Gardenia jasminoides, Gentiana scabra root extract, berberine from Berberis asiatica root, fenugreek seed extract, kalonji seed extract, black pepper extract, and methanol extract of Coreopsis lanceolata.

19. The formulation of claim 18 wherein said pomegranate extract is from about 50 mg to about 300 mg, said glutamine is from about 100 mg to about 500 mg, said ginsenosides is from about 50 mg to about 200 mg, said curcumin is from about 50 mg to about 300 mg, said fruit extract of Gardenia jasminoides is from about 50 mg to about 300 mg, said Gentiana scabra root extract is from about 25 mg to about 200 mg, said berberineis from about 50 mg to about 300 mg, said fenugreek seed extract is from about 100 mg to about 400 mg, said kalonji seed extract is from about 100 mg to about 400 mg, said black pepper extract is from about 5 mg to 10 mg, and said methanol extract of Coreopsis lanceolata is from about 50 micrograms to about 200 micrograms.

20. The formulation of claim 18 further comprises at least one carrier, said carrier is selected from the group consisting of: powder, liquid, pill, hard shell capsule, soft shell capsule, gel, tablet, ointment, solution and combinations thereof.
